(19) United States
(12) Patent Application Publication
(10) Pub. No.: US 2017/0070654 A1
(43) Pub. Date: Mar. 9, 2017

(54) IMAGING NEEDLE APPARATUS

(71) Applicant: Samark Technology LLC, Sarasota, FL (US)

(72) Inventors: Sam Seiichiro OCHI, Lakewood Ranch, FL (US); Mark WALTER, Sarasota, FL (US)

(21) Appl. No.: 15/261,743

(22) Filed: Sep. 9, 2016

Related U.S. Application Data
(60) Provisional application No. 62/216,334, filed on Sep. 9, 2015.

(51) Int. Cl. H04N 5/225; H04N 9/04; A61B 1/06; G02B 23/24; A61B 1/317; A61B 1/04; H04N 5/374; G02B 23/26

(52) U.S. Cl. CPC H04N 5/2256; H04N 5/374; H04N 9/045; H04N 5/2254; G02B 23/26; G02B 23/2461; A61B 1/317; A61B 1/042; A61B 1/06; H04N 2005/2255

(57) ABSTRACT

An imager includes a lens assembly, an imager chip, and a light pipe assembly. The lens assembly may include a plurality of lenses, the imager chip may include a plurality of photocells, and the light pipe assembly may include a plurality of light pipes optically coupled between the plurality of lenses and the plurality of image sensors.

### CROSS REFERENCE TO RELATED APPLICATIONS

This application claims the benefit of U.S. Provisional Patent Application No. 62/216,334, filed on Sep. 9, 2015, which is incorporated by reference herein for all purposes.

### BACKGROUND

Traditional surgical procedures are open procedures. In an open surgical procedure, a surgeon makes a large incision on a patient in order to view and correct physical ailments using surgical tools. Open procedures have several drawbacks. The large surgical incisions used to perform open procedures can become infected. Surgeons may damage surrounding tissues during open procedures, while trying to manipulate the surgical site. Open procedures often require patients to undergo full anesthesia, which independently increases risks of death and/or serious complications. In addition, open procedures can cause patients severe discomfort during recovery periods.

In order to avoid the complications of open procedures, surgeons have developed minimally invasive surgical techniques to perform surgeries that were traditionally performed as open procedures. In contrast to open procedures, minimally invasive procedures can be performed by inserting surgical tools through small incisions in a patient's skin. Minimally invasive procedures have various advantages over open procedures, including lower infection risks, lower patient discomfort, and lower anesthesia requirements.

The small incisions used in minimally invasive procedures make viewing the surgical field difficult. Accordingly, surgeons generally use imaging devices, e.g., endoscopes, during minimally invasive procedures in order to indirectly view the surgical field. Some of these imaging devices must be inserted into a patient's body through the small incisions.

Arthroscopy is a type of minimally invasive orthopedic procedure performed in a skeletal joint cavity. An arthroscope includes a camera that may be inserted directly into a skeletal joint. With help from the arthroscope, surgeons can diagnose various problems related to the skeletal joint.

In certain cases, arthroscopes can be used to determine whether a therapeutic material should be delivered to the skeletal joint. For example, a surgeon may use an arthroscope to determine whether to deliver a drug, stem cells, or anesthesia for a future procedure to the skeletal joint. Some of these therapeutic materials can be injected using a syringe and a needle.

The cameras used in medical applications, such as those used in arthroscopes, should be precise and have high resolution. However, traditional high-resolution imaging modalities may be expensive. Furthermore, these imaging modalities may be bulky, and unsuitable for certain applications.

### BRIEF SUMMARY OF THE DISCLOSURE

An imager includes a lens assembly, an imager chip, and a light pipe assembly. The lens assembly may include a plurality of lenses, the imager chip may include a plurality of photocells, and the light pipe assembly may include a plurality of light pipes optically coupled between the plurality of lenses and the plurality of image sensors.

The lens assembly may further include a plurality of dividers between the plurality of lenses, the plurality of dividers including an optically opaque material.

Each of the plurality of lenses may include a stack of transparent layers. The transparent layers may be stacked in an order of decreasing size.

Each of the plurality of lenses may bend light at a predetermined angle and focus light at one of the plurality of photocells. A first lens at an edge of the light pipe assembly may bend light at a greater angle than a second lens at a center of the light pipe assembly.

The plurality of photocells may be spaced apart from the plurality of lenses by a focal distance of the plurality of lenses.

The plurality of photocells may include complementary metal oxide semiconductor (CMOS) image sensors.

The light pipe assembly may further include an isolation material disposed between the light pipes.

Each of the plurality of light pipes may include a core material surrounded by a sheath material, the core material having a lower index of refraction than the sheath material.

The imager chip may further include a plurality of pixels, each of the plurality of pixels being divided into a plurality of color areas, and each of the photocells may correspond to at least one of the color areas.

Each of the plurality of light pipes may be coupled to at least one of the photocells.

An image device may include a needle, an imager, and a syringe coupled to the needle. The needle may include a tip. The imager may be disposed on the needle, and may include a lens assembly, an imager chip, and a light pipe assembly. The lens assembly may include a plurality of lenses, the imager chip may include a plurality of image sensors, and the light pipe assembly may include a plurality of light pipes optically coupled between the plurality of lenses and the plurality of image sensors.

The imaging device may further include a light-emitting diode (LED) provided proximate to the imager; and a bypass capacitor disposed inside of the needle and proximate to the imager.

The lens assembly may further include a plurality of dividers between the plurality of lenses, the plurality of dividers including an optically opaque material, wherein each of the plurality of lenses includes a stack of transparent layers, the transparent layers being stacked in an order of decreasing size.

Each of the plurality of lenses may bend and focus light toward one of the plurality of photocells, wherein a first lens at an edge of the light pipe assembly bends light at a greater angle than a second lens at a center of the light pipe assembly.

The plurality of image sensors may be spaced apart from the plurality of lenses by a focal distance of the plurality of lenses.

The light pipe assembly may further include an isolation material disposed between the light pipes, wherein each of the plurality of light pipes includes a core material surrounded by a sheath material, the sheath material having a larger index of refraction than the core material.

The imager chip may further include a plurality of pixels, each of the plurality of pixels being divided into a plurality of color areas. Each of the photocells may correspond to at least one of the color areas, and each of the plurality of light pipes may be coupled to at least one of the photocells.

### BRIEF DESCRIPTION OF THE DRAWINGS

FIG. 1 illustrates an imaging apparatus according to an embodiment.

FIG. 2 illustrates a needle the imaging apparatus from a first side view according to an embodiment.

FIG. 3 illustrates the needle of the imaging apparatus from the second view according to an embodiment.

FIG. 4 illustrates a cross-section of the needle of the imaging apparatus along a line A-A′ according to an embodiment.

FIG. 5 illustrates a cross-section of the needle of the imaging apparatus along a line B-B′ according to an embodiment.

FIG. 6 illustrates a cross-section of the needle of the imaging apparatus along a line C-C′ according to an embodiment.

FIG. 7 illustrates a cross-section of the needle of the imaging apparatus along a line D-D′ according to an embodiment.

FIG. 8 illustrates a top view, a left or right side view, and a lower side view of a bee-eye imager according to an embodiment.

FIG. 9 illustrates a side view and partial cross sections of a bee-eye imager according to an embodiment.

FIG. 10 illustrates a cross section of a bee-eye imager according to an embodiment.

FIGS. 11A through 11I illustrate fabrication steps of a bee-eye imager according to an embodiment.

FIG. 12 illustrates a detail view of a bee-eye light pipe according to an embodiment.

FIG. 13 illustrates a detail view of a bee-eye imager pixel according to an embodiment.

FIG. 14 illustrates a detail view of a bee-eye imager pixel according to an embodiment.

FIG. 15 illustrates a detail view of a bee-eye imager pixel according to an embodiment.

FIG. 16 illustrates a detail view of a bee-eye imager pixel according to an embodiment.

FIG. 17 illustrates a detail view of a bee-eye imager pixel according to an embodiment.

FIG. 18 illustrates horizontal and vertical fields of view of a bee-eye imager according to an embodiment.

FIG. 19 illustrates a linear pixel array of a bee eye imager, according to an embodiment.

FIG. 20 illustrates one-dimensional pixel photocell targets of a bee-eye imager according to an embodiment.

FIG. 21 illustrates pixel positioning nomenclature according to an embodiment.

FIG. 22 illustrates an imager and a lens assembly according to an embodiment.

### DETAILED DESCRIPTION OF THE DISCLOSURE

A detailed description of embodiments is provided below along with accompanying figures. The scope of this disclosure is limited only by the claims and encompasses numerous alternatives, modifications and equivalents. Although steps of various processes are presented in a particular order, embodiments are not necessarily limited to being performed in the listed order. In some embodiments, certain operations may be performed simultaneously, in an order other than the described order, or not performed at all.

Numerous specific details are set forth in the following description in order to provide a thorough understanding of the present disclosure. These details are provided for the purpose of example and embodiments may be practiced according to the claims without some or all of these specific details. For the purpose of clarity, technical material that is known in the technical fields related to this disclosure has not been described in detail so that the disclosure is not unnecessarily obscured.

The present disclosure relates to an imaging needle apparatus and a bee-eye imager. The imaging needle apparatus can take pictures, or video, or both using an image capturing device or imager. The image capturing device or imager may be proximately located to a needle. In an embodiment, the apparatus is a video syringe and is capable of capturing images and injecting fluid into a desired location, e.g., at a knee or shoulder joint of a person. In an embodiment, the fluid may be stem cell fluid, drugs, or other fluid used for medical treatment. The imaging needle apparatus may capture images and/or video using the bee-eye imager.

In an embodiment, the bee-eye imager includes a lens system that has a concave configuration globally and a convex configuration locally. The lens system uses the Fresnel lens concept for each lens focused onto an individual pixel center. The global concave nature of the lens system allows for a large field of view, for example, greater than 60 degrees along at least one axis. The concave nature of the individual lens allows for an approximate focusing distance, such as about 100 micrometers. Below are some of the concepts associated with the lens system according to an embodiment.

An image may be restricted from an individual lens to a photocell area in an individual pixel. For example, a 7.5 micrometer diameter individual lens may restrict light to a 2 um diameter light sensitive photocell area of an 8 micrometer by 8 micrometer pixel. The focus of the lens may be within the photocell area. Any light that goes through the lens and falls outside the photocell area may not affect this light sensitive photocell.

The size of the light-sensitive photocell at the pixel center may be adjusted to increase resolution. The smaller the size of the photocell area, the greater the resolution. However, the efficiency of the light sensitive photocell diode may drop as the dimensions approach the wavelength of light at, for example, at 0.5 micrometers.

The resolution of the bee-eye imager may also depend on the focal length of the lens assembly. The resolution may increase as the focal length increases. Accordingly, the focal length of the bee-eye imager may be increased as size and space permits. The focal length may be adjusted to be greater than 100 micrometers, for example.

The lenses of the bee-eye imager may be focused to infinity. Accordingly, the bee-eye imager can be made without expensive and/or bulky lenses. The bee-eye imager can be built into small devices, such as imaging needles and drones the size of insects.

The bee-eye imager can be implemented using standard wafer stacking and 3D assembly technology, such as techniques used in the universal service bus (USB) memory industry. A top level of the bee-eye imager may be the lens assembly. The next level may be a light pipe assembly, which may have a thickness between 100 micrometers and 400 micrometers. The greater the thickness of the light pipe assembly, the greater the resolution. An imager chip may be disposed underneath the light pipe assembly. The imager chip may be a back-lit imager chip or a standard front-lit imager. The imager chip may include CMOS circuitry built adjacent to the photo diode cell to improve sensitivity and pre-processing, and to reduce complexity and power further down the image processing chain. Other configurations are possible.

The imager chip may include circuitry that pre-processes captured images. The imager chip may have reduced power overhead to minimize power dissipation within the imager chip and circuitry surrounding the imager chip. The captured images may be converted to USB compatible data.

The pre-processing circuitry may be integrated into individual pixels, and may provide for image-stitching. A “mosaic image” may be generated by stitching images together so that a wider field of view is created as new image data is provided by the imager chip. This creates an image wider and greater than the imager chip could otherwise provide.

The bee-eye imager may be provided on a needle of a video syringe. As the needle of the video syringe moves toward its target and provides instantaneous image updates, each of the previous images may be retained in memory and processed to create a wider field of view. The video syringe may recognize that the data viewed is “still” with respect to itself. The subject area viewed is looked upon as stationary—except the information originating from the video syringe. Using processing circuitry in the bee-eye imager, components of an old frame can be reused in a new frame, when the components of the old frame are stationary with respect to new frame. An old image can be “morphed” to accommodate a new image. The video syringe may also take advantage of the fact that most of the movement of the needle may be vertical and not horizontal (with respect to the shaft of the needle). This may be somewhat like a “magic” paint brush that creates a picture as the brush is manipulated over a canvas.

A standardized image of an area being examined by the bee-eye imager may be displayed by a monitor with a current image and/or video frame, such that a user can compare features, e.g., color, shape, etc. In an embodiment, the images and/or video output by the bee-eye imager may be recorded.

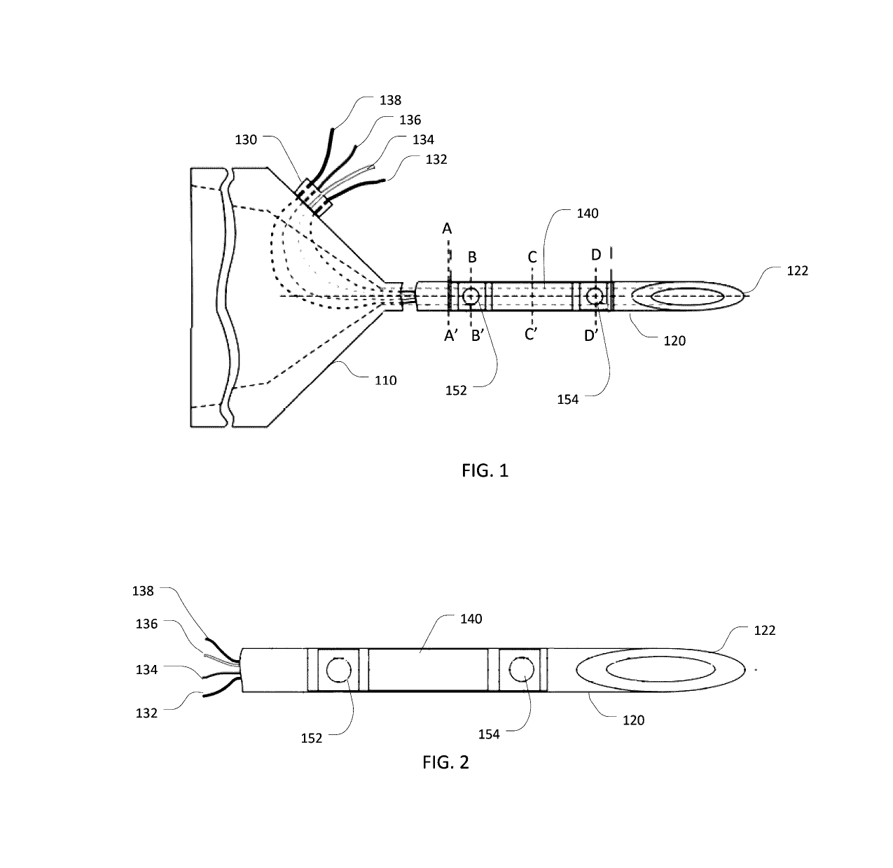

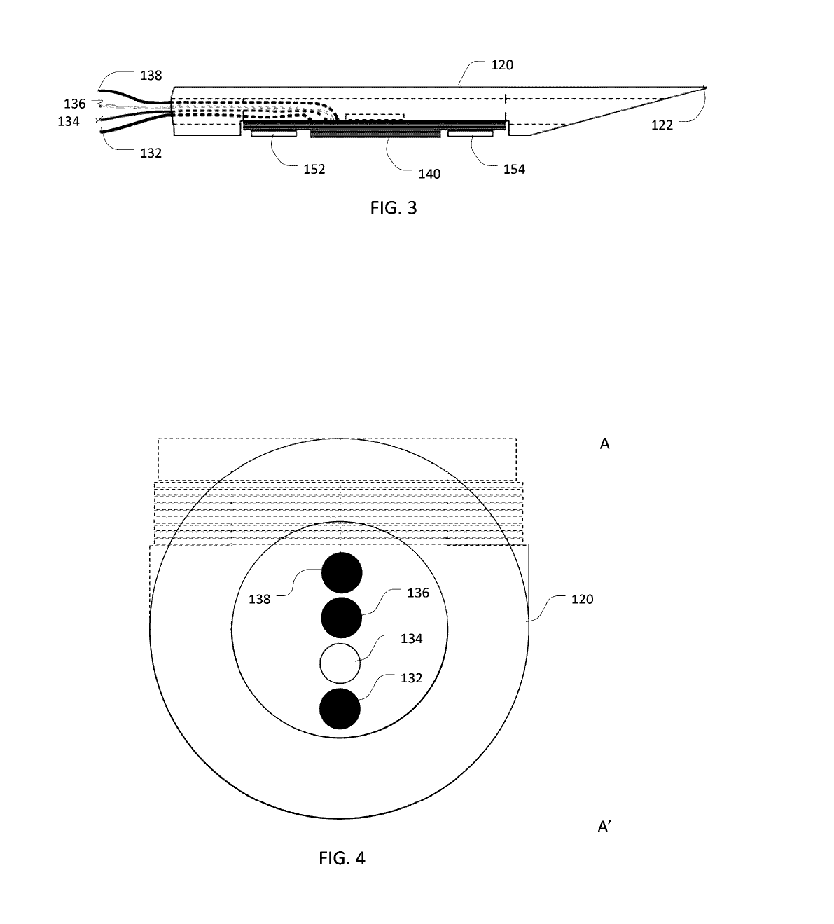

FIG. 1 shows an imaging apparatus 100 according to an embodiment of the present disclosure. The imaging apparatus 100 may include a syringe 110, a needle 120, a wire output 130, an imager 140, and first and second light-emitting diodes (LEDs) 152 and 154. FIGS. 2 and 3 illustrate the needle 120 from first and second side views according to an embodiment of the present disclosure.

The syringe 110 may be used to inject fluid through the needle 120. The syringe 110 may be hollow, and may stably house fluid before the fluid is injected through the needle 120. The syringe 110 may exert a positive pressure on the fluid in order to propel the fluid through the needle 120. The syringe 110 may push fluid in a direction that is parallel to the needle 120, such that the fluid can be propelled through the needle 120 with relatively laminar flow. That is, the position of the syringe 110 with respect to the needle 120 prevents fluids from turbulently flowing through the needle 120.

The syringe 110 may include a plunger or a pump that may propel the fluid through the needle 120. When the syringe 110 includes a plunger, a user can deliver fluid through the needle 120 by pushing the plunger toward the needle 120 in a direction parallel to the needle 120.

The needle 120 may include a sharp tip 122 that can pierce soft tissue. Although not illustrated, the sharp tip 122 may be configured to be retractable into the needle 120 so that the needle 120 would have a blunt tip (not shown) when the sharp tip 122 is retracted into the needle 120. In an embodiment, the needle 120 can pierce soft tissue around a skeletal joint, such as a knee.

As shown by FIG. 3, the tip 122 of the needle 120 may be tapered. The needle 120 may be a hypodermic needle. The tip 122 may be comprised of stainless steel. In an embodiment, an outer diameter of the needle may be between 0.0280 inches and 0.0285 inches, and an inner diameter of the needle may be between 0.0155 and 0.0175 inches. The needle 120 may be a 22-gauge hypodermic needle. In an embodiment, the needle 120 includes a flat outer surface attached to the imager 140. The imager 140 may be placed in other locations, e.g., on the front of the needle 120.

A fluid path may be provided within the needle 120 for fluid that may be injected into a specific site. The fluid may include, e.g., a fluid drug or stem cell fluid. When the syringe 110 increases pressure in the fluid path, the fluid can be emitted from the tip 122 of the needle 120.

In an embodiment, the needle 120 includes a first path for the wires and a second, separate path for injecting fluid. In another embodiment, the needle 120 has a single path shared by output wires from the imager 140 and the fluid.

The needle 120 may be attached or detached from the syringe 110. In an embodiment, the bayonet of the needle 120 may screw onto threads disposed on an attachment point of the syringe 110.

The wire output 130 may be coupled to one or more conductive wires that output imaging data from the imager 140. The wire output 130 may be further coupled to one or more wires that supply power to the imager 140. In other embodiments, however, fewer or additional wires may be coupled to the wire output 130.

The wire output 130 may be coupled to a battery, and may transfer power from the battery to the imager. In an embodiment, the wire output 130 transfers power to the imager from an external device, such as an external display device, via a wired or wireless connection.

The wire output 130 may include one or more processors that convert the imaging data from the imager 140 to a standard video format. In an embodiment, the wire output 130 receives imaging data from the imager 140. The imaging data may be in the standard video format, e.g., Universal Serial Bus (USB) or High-Definition Multimedia Interface (HDMI) compatible.

The wire output 130 may convert one or more of the wires transferring imaging data and/or power to and from the imager 140 into a single output wire. In an embodiment, the wire output 130 may output the one or more signal and power wires to a single socket or plug that may interface with an external display device. The socket or plug may be a USB or an HDMI socket or plug, or other communication interfaces. The external display device may thus display imaging data from the imager 140 and may power the imager 140 via the wire output 130.

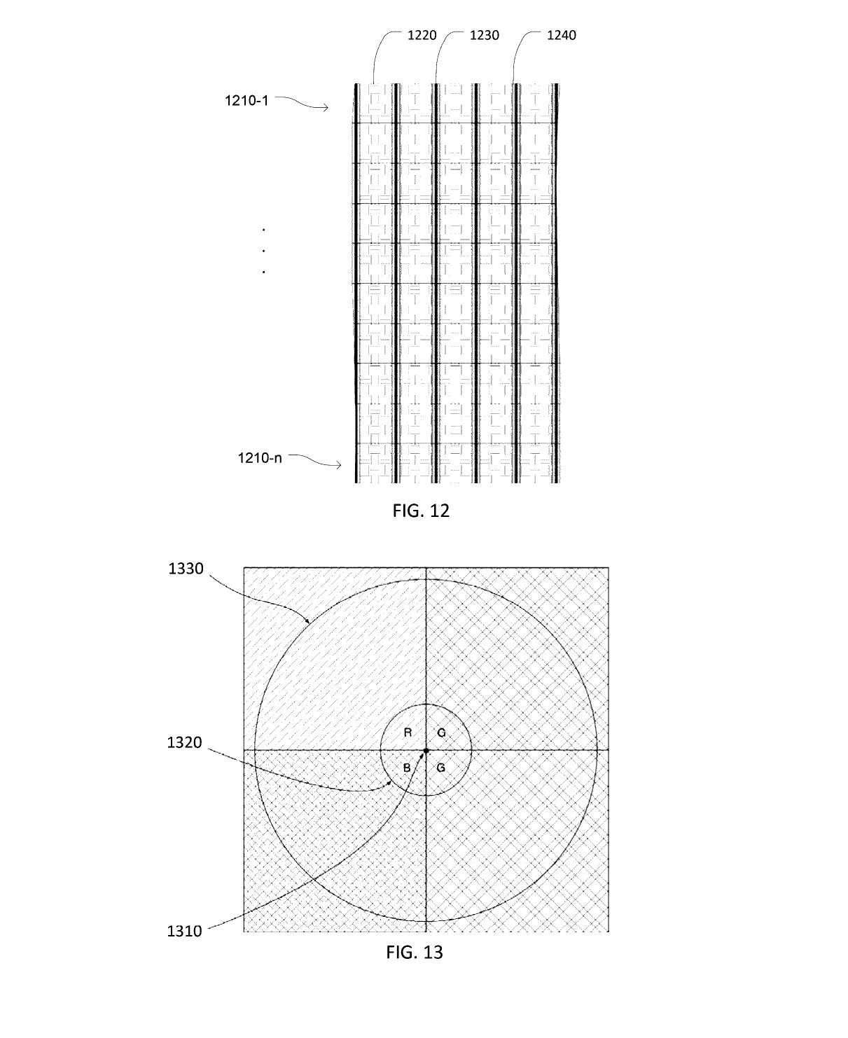

In an embodiment, the imaging needle apparatus includes or is coupled to a communication component (not shown) that wirelessly transmits the imaging data to an external display device 1220 (see FIG. 12) using Bluetooth or Wi-Fi, or other wireless communication protocols.

Referring back to FIG. 1, four wires are coupled between the wire output 130 and the imager 140. The wires may include a positive power wire 132, a negative power wire 134, a positive data wire 136, and a negative data wire 138.

Each of the wires 132, 134, 136, and 138 may include a conductive material and an insulative material that covers the conductive material. In an embodiment, each wire has a diameter of about 75 μm. The wires may be color coded: for example, the positive power wire 132 may be red, the positive data wire 136 may be white, the negative data wire 138 may be green, and the negative power wire 134 may be black.

One or more of the wires 132, 134, 136, and 138 may be micro-coaxial wires, as discussed below with respect to FIGS. 8 through 11. In an embodiment, all of the wires 132, 134, 136, and 138 are integrated into a single micro-coaxial wire with a data bypass capacitor and a power supply bypass capacitor.

The wires 132, 134, 136, and 138 may protrude from the imager 140 into the interior of the needle 120. The wires 132, 134, 136, and 138 may be threaded within the interior of the needle 120, pass through a bayonet of the needle 120, and disposed in an interior space of the syringe 110, and attach to an interior side of the wire output 130. The wires 132, 134, 136, and 138 may be electrically coupled between the imager 140 and the wire output 130.

The imager 140 may be used to capture images and/or video of spaces and structures disposed in the vicinity of the needle 120. The imager 140 may capture images and/or video in one or more directions with respect to the needle 120. The imager 140 may, for example, capture images and/or video in a radial direction with respect to the needle 120. The imager 140 may output the images and/or video as imaging data.

The imager 140 may be fixed at a position proximate to the needle 120. For example, the imager 140 may be attached to an outer surface of the needle 120. The imager 140 may be attached to the outer surface of the needle 120 with an adhesive. In an embodiment, the imager 140 is fixed on an outside surface of the needle 120 at a position within ⅛ to ½ inches of the tip 122. In an embodiment, the imager 140 may be attached to a flat exterior surface of the needle 120.

In an embodiment, the imager 140 is a bee-eye imager including a plurality of complementary metal-oxide semiconductor (CMOS) pixels. The imager 140 may include a plurality of image sensors corresponding to a plurality of pixels. The image sensors may be a plurality of imaging chips disposed along a flat surface, for example.

The imager 140 may include one or more controllers coupled between the communication interface and the circuits in the rest of the imager 140. The imager 140 may further include analog and digital control electronics that convert imaging signals from the imager 140 to signals compatible with a communication interface. The imager 140 may include electronics that converts raw imaging data to imaging data that is compatible with the wire output 130 and/or external display device. For example, the imager 140 may convert the raw imaging data to uncompressed video imaging data compatible with a USB- or HDMI-based interface.

The imager 140 may output data to a first bypass capacitor that is coupled between the imager 140 and the positive and negative data wires 136 and 138. In addition, a second bypass capacitor may be coupled between the imager 140 and the positive and negative data wires 132 and 134. The first and second bypass capacitors may reduce noise in signals transmitted through the wires 132, 134, 136, and 138.

The first and second LEDs 152 and 154 may illuminate areas around the needle 120, in order to more easily capture high quality images using the imager 140. In an embodiment, one or both of the first and second LEDs 152 and 154 emits white light. The first and second LEDs 152 and 154 may be integrated into the imager 140. In an embodiment, the first and second LEDs 152 and 154 each emit light from a surface having an area of 680 μm by 680 μm.

In an embodiment, the first LED 152 may be located between the imager 140 and the syringe 130. The second LED may be located between the imager 140 and the tip 122 of the needle 120.

Although two LEDs are illustrated, embodiments are not so limited. The apparatus 100 may include more or fewer than two LEDs.

The imager 140 and the first and second LEDs 152 and 154 may be covered with a clear, biocompatible sealant (not shown). The sealant may fix the imager 140 and the first and second LEDs 152 and 154 onto the needle 120, such that components of the imager 140 and the first and second LEDs 152 and 152 do not become dislodged, e.g., by human tissue, when the imaging apparatus 100 is used during a medical procedure. In addition, the sealant may cover the imager 140 and the first and second LEDs 152 and 154 with a smooth surface, such that the needle 120 can be smoothly inserted into a desired site.

FIGS. 4 through 7 show cross sections of the needle 140 according to an embodiment of the present disclosure.

FIG. 4 illustrates a cross section of the needle 120 between the syringe 130 and he imager 140 along a line A-A′ illustrated in FIG. 1. FIG. 4 shows that the outer surface of the needle 120 may be rounded where the imager 140 is not present.

FIG. 5 shows a cross section of the needle 120, the first LED 152, the imager 140, and the wires 132, 134, 136, and 138 along a line B-B′ illustrated in FIG. 1.

The first LED 152 may be disposed on the imager 140. The first LED 152 may emit light in a direction that points away from the needle 120 and in an imaging direction of the imager 140.

The imager 140 may be disposed between the first LED and the needle 120. The imager 140 may include a plurality of stacked chips. The stacked chips of the imager 140 may include an imaging chip 142 and an integrated circuit (IC) stack 144. The IC stack 144 may include, for example, silicon CMOS circuits. Each stacked chip may have a thickness of approximately 10 μm or less. In an embodiment, one or more stacked chips are each 5-8 μm thick. The imaging chip 142 may be located on top of the stack of control ICs 144. The number of ICs in IC stack 144 may vary depending on the implementation.

The first and second LEDs 152 and 154 as well as the first stack, may be located above the second stack of the imager 140. That is, the first and second LEDs 152 and 154 may be stacked on the imaging chip 142.

In an embodiment, the first LED 152 and the imager 140 are flat structures and may be attached to an outer surface of the needle 120. Accordingly, the device 100 may have a flat side where the imager 140 is attached to the outer surface of the needle 120, even though rest of the outer surface of the needle 120 may be curved.

In an embodiment, the outer corners of the imager 140 may be rounded, such that the needle 120 and the imager 140 together provide a round, elongated shape resembling a conventional, cylindrical needle. In an embodiment, one or more of control ICs in the imager 140 may fixed to an interior surface of the needle 120, such that the imager 140 may be partially disposed inside of the needle 120.

The wires 132, 134, 136, and 138 may extend from the imager 140 into the interior of the needle 120 and underneath the imager 140 and the first LED 152.

As shown in FIGS. 4 and 5, the wires 132, 134, 136, and 138 may have small cross-sectional areas compared to the interior of the needle, which may provide ample interior space for fluids to be injected from the tip 122 of the needle 120 with substantially laminar flow. In an embodiment, the cross-sectional area of the wires 132, 134, 136, and 138 may take up between 5 and 25% of the interior cross-sectional area of the needle 120.

In an embodiment, the wires 132, 134, 136, and 138 may be fixed to an interior surface of the needle 120, in order to provide a more continuous fluid path through the needle 120. The wires 132, 134, 136, and 138 may be glued to the interior surface of the needle 120.

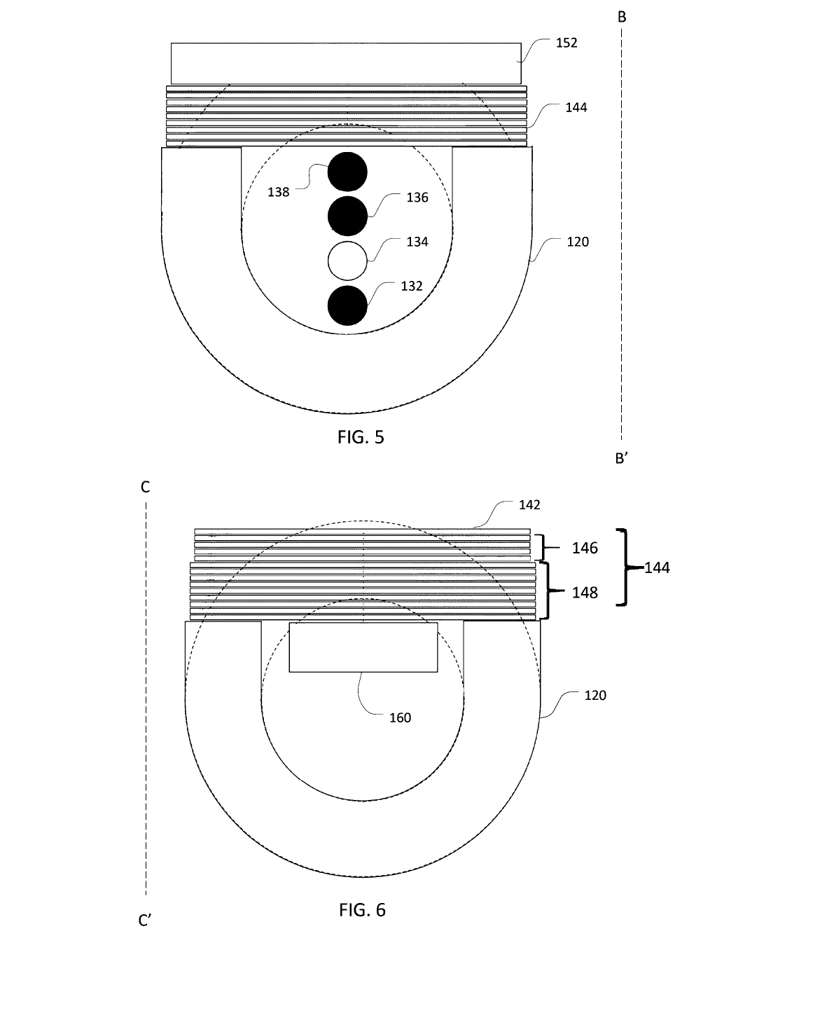

FIG. 6 illustrates a cross-section of the needle 120, the imager 140, and a bypass capacitor 160 along a line C-C′ according to an embodiment of the present disclosure. Specifically, FIG. 6 shows a cross-section of the imaging chip 142 and the IC stack 144 of the imager 140.

The imaging chip 142 may be disposed on top of the IC stack 144. The imaging chip 142 may include a plurality of imaging sensors corresponding to pixels. In an embodiment, the imaging chip 142 is a 1.36 mega pixel imager, and may include a 1 μm² pixel array located over an area of 680 μm by 2000 μm on top of the IC stack 144.

The imaging chip 142 may convert image signals into electrical signals, and may pass the electrical signals to circuitry in the IC stack 144.

The IC stack 144 may include a plurality of ICs. In an embodiment, the plurality of ICs in the IC stack 144 may be divided into a first plurality of ICs in a first stack 146 and a second plurality of ICs in a second stack 148. Each of the plurality of ICs in the IC stack 144 may include a silicon wafer.

The first stack 146 may be stacked on top of the second stack 148, between the imaging chip 142 and the second stack 148. In an embodiment, the first stack 146 may include a stack of four CMOS ICs. In an embodiment, each of the CMOS ICs has a stacking surface with an area of a first size, for example, 680 μm by 2000 μm.

The second stack 148 may be disposed between the bypass capacitor 160 and the first stack 146. In an embodiment, the second stack 148 includes a first logic layer, a plurality of bit layers, and a second logic layer. The first logic layer (the “controller”) may contain sense amps, write drivers, address decoders, and other elements that read and write memory bits. The plurality of bit layers may be stacked on the first logic layer. The second logic layer (the “I/O layer”) may be attached underneath the controller and the plurality of bit layers. The I/O layer translates a signal from the controller according to a voltage and protocol that is understandable by an off-chip device, such as a processor, a field-programmable gate array (FPGA), an optical link, or other device.

In an embodiment, the second stack 148 may include 9 CMOS ICs. In an embodiment, each of the CMOS ICs has a stacking surface with an area of a second size that is larger than the first size, for example, 710 μm by 4000 μm. Alternatively, the second stack 148 may include 9 CMOS ICs that may each have a surface with an area of 700 μm by 4000 μm.

The second stack 148 may therefore have a larger width than the first stack 146 and the imaging chip 142. In addition, the second stack 148 may have a larger length than the first stack 146 or the imaging chip 142. Accordingly, the outer surface of the imager 140 may have slightly rounded edges, so that the needle 120 may be smoothly inserted into a desired surgical site.

In an embodiment, the first stack 146 and the imaging chip 142 may have substantially the same height as each of the first and second LEDs 152 and 154. A surgical-grade material may be disposed between the first stack 146 and the imaging chip 142 and the first and second LEDs 152 and 154, so that the apparatus 100 has a smooth outer surface.

The plurality of ICs in the IC stack 144 may be interconnected by through silicon vias (TSVs) and contacts. In some embodiments, the IC stack 144 and the imaging chip 142 may include copper TSVs and contacts. Alternatively or additionally, the IC stack 144 and the imaging chip 142 may include tungsten TSVs and contacts.

Tungsten TSVs and contacts provide a number of advantages over copper TSVs and contacts. Advantages of ICs with tungsten include better thermal compatibility, size, and density than ICs with copper alone.

Tungsten is more thermally compatible with silicon than copper. Tungsten and silicon have similar coefficients of thermal expansion. Accordingly, tungsten TSVs and contacts apply limited physical distress on surrounding a silicon wafer during operating conditions.

In addition, tungsten structures may be smaller than copper structures, and may therefore may have almost negligible inductance, capacitance, and resistance. Accordingly, tungsten contacts may be more cheap and reliable than copper contacts. For example, the tungsten contacts in the IC stack 144 may fill a 10 μm deep hole with a 10:1 aspect ratio. In an embodiment, the tungsten TSVs can have diameters of 1 μm or less, even with a 10:1 aspect ratio limit, when a wafer thickness is 10 μm or less. In an embodiment, the tungsten contacts may also be 1 μm (or less) wide, and arrayed on a 2 μm or smaller pitch (the center to center distance between repeated objects). Thus, embodiment of the IC stack 144 may include tungsten TSVs and contacts instead of larger copper through silicon vias (TSVs).

In contrast, copper TSVs may have a larger width than tungsten TSVs. For example, the copper TSVs in the IC stack 144 may be 5 μm wide and located on a 40 to 50 μm pitch. Copper is less thermally compatible with silicon than tungsten. That is, copper has a different thermal coefficient of expansion than silicon.

Tungsten can also be used to fabricate a more densely connected IC than copper alone. In an embodiment, a wafer in the IC stack 144 includes 5 μm wide copper TSVs spaced on a grid of 40 or more μm per step, and may alternatively or additionally include tungsten contacts can be organized on a pitch that is about two times the contact diameter, (e.g. 0.6 μm wide contacts can be on a 1.2 μm pitch, and 1 μm wide contacts can be on a 2 μm pitch). Thus, the tungsten contacts in the IC stack 144 may be made with very small diameters, and can also be arrayed on a very tight pitch. Thus, tungsten TSVs and contacts support a higher vertical interconnect per unit area across the surface of each wafer in the IC stack 144 than copper TSVs and contacts, and therefore support higher interconnect.

In an embodiment, the wafers of the IC stack 144 include only tungsten TSVs or contacts, or only a limited number of copper TSVs and contacts. In an embodiment, when a wafer of the IC stack 144 includes too much copper, a normal temperature change can break the wafer. Even if the die or wafer does not break, if a transistor is located too close to the copper TSV, the expansion or contraction of the copper can change the operating characteristics of the transistor, and may make the rest of the IC non-functional.

Due to high vertical interconnect from tungsten and copper TSVs and contacts, it is possible to perform potent and comprehensive post-assembly repair of the ICs in the IC stack 144. In an embodiment, a variety of redundant circuit elements are available, including spare contacts. In addition, redundant elements from one die may be used to repair defects in another die in the IC stack 144. The IC stack 144 may become more reparable by adding more dies to the stack.

That is, because the IC stack 144 may include small tungsten TSVs rather than large copper TSVs as vertical interconnects, the IC stack 144 may support post-assembly repair. Connections throughout the IC stack 144 may be located in precise locations, and there may be enough connections to do thorough post-assembly repair. Thus, the IC stack 144 of the imager 140 may include a memory subsystem that has high density, performance, and that operates under low power.

The bypass capacitor 160 may be disposed underneath the imager 140 inside of the interior space of the needle 120. The bypass capacitor 160 may be coupled between the second stack 148 of the imager 140 and the wires 132, 134, 136, and 138, and may reduce noise in imaging data transmitted by the positive data wire 136 and the negative data wire 138. One terminal of the bypass capacitor 160 may be connected to the positive data wire 136, and a second terminal of the bypass capacitor 160 may be connected to the negative data wire 138.

The bypass capacitor 160 may be of a 0201 size, leaving a cross sectional area for injected fluids within the interior of the needle 120. Fluids may flow through the needle 120, unimpeded by the imager 140 including the first and second stacks.

In an embodiment, another bypass capacitor may be disposed underneath the imager 140, and may also reduce noise in power supplied to the imager 140 through the positive power wire 132 and the negative power wire 134. The other bypass capacitor can be coupled between the positive power wire 132 and the negative power wire 134.

Embodiments of the apparatus 100 may be manufactured at a low cost. As such, the apparatus 100 can be a single use, disposable device. The video syringe can be a cost-effective alternative to conventional arthroscopes, for example.

The control ICs among the IC stack 144 and the imaging chip 142 may be manufactured using one or more of the following manufacturing methods.

A high performance CMOS process may be used to build high performance logic circuits in the imager 140, such as sense amps, write drivers, and decoders. A dynamic random-access memory (DRAM) process can be used to build memory storage bits in the imager 140. Larger feature-size processes can be used when they offer the right capabilities at a good cost, and more expensive, advanced processes can be used when they are needed. This mix of cheaper and more expensive processes can be used to build a single, highly optimized device.

Embodiments of the imaging needle apparatus include one or more wires that connect an imager to external electronics, such as a display apparatus. As noted above, in order to reduce the effect of noise across the one or more wires, each of the wires may be coupled to the bypass capacitor 160. In an embodiment, each wire is a micro-coaxial wire, in which the bypass capacitor is incorporated into the wire itself.

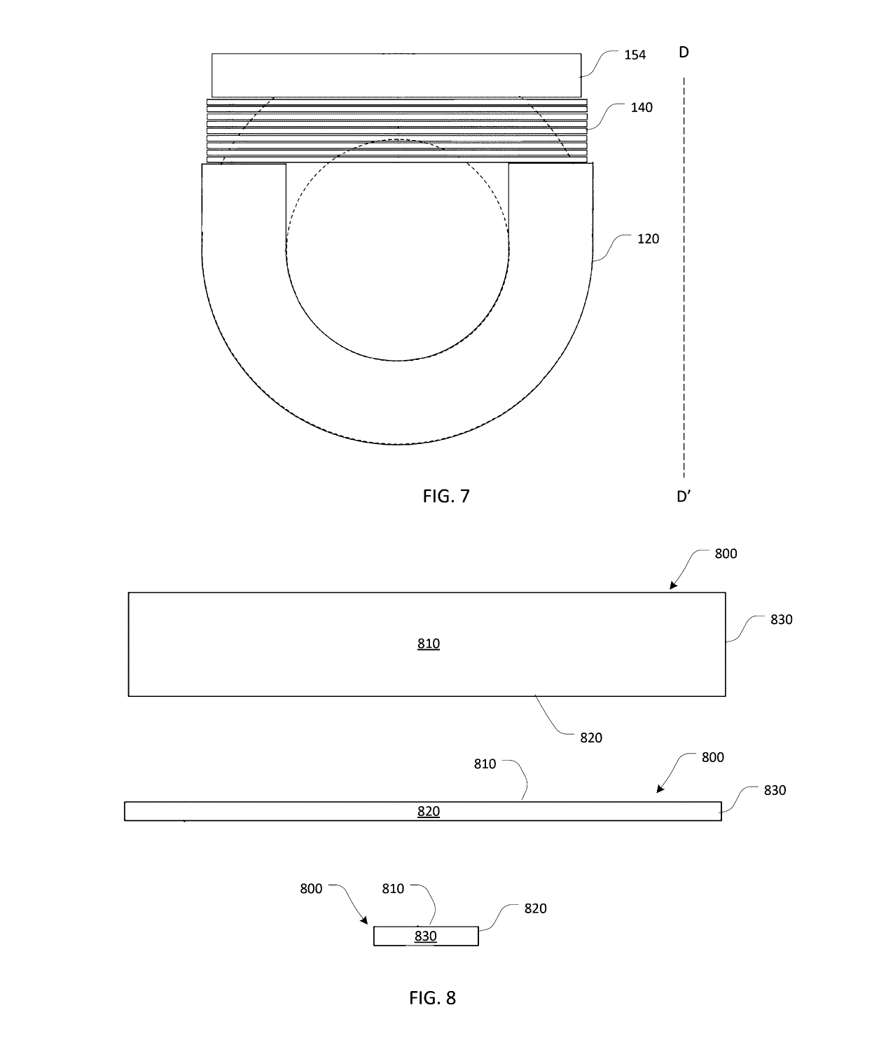

FIG. 7 illustrates a cross-section of the needle 120, imager 140, and the first LED 154 along a line D-D′ illustrated in FIG. 1.

FIG. 8 illustrates a top view, a left or right side view, and a lower side view of a bee-eye imager 800, according to an embodiment. The bee-eye imager 800 may have the shape of a rectangular prism.

The bee-eye imager 800 may include a first side 810, a second side 820, and a third side 830. The first side 810 may point radially from a structure on which the bee-eye imager 800 is mounted, and may receive light to be imaged. In an embodiment, the first side 810 has a length of 4 mm, and a width of 0.7 mm.

The second side 820 may be perpendicular to the first side 810. In an embodiment, the second side has a length of 4 mm and a width of 126 micrometers.

The third side 830 may be perpendicular to the first side 810 and the second side 820. In an embodiment, the third side 830 has a length of 0.7 mm and a width of 126 micrometers.

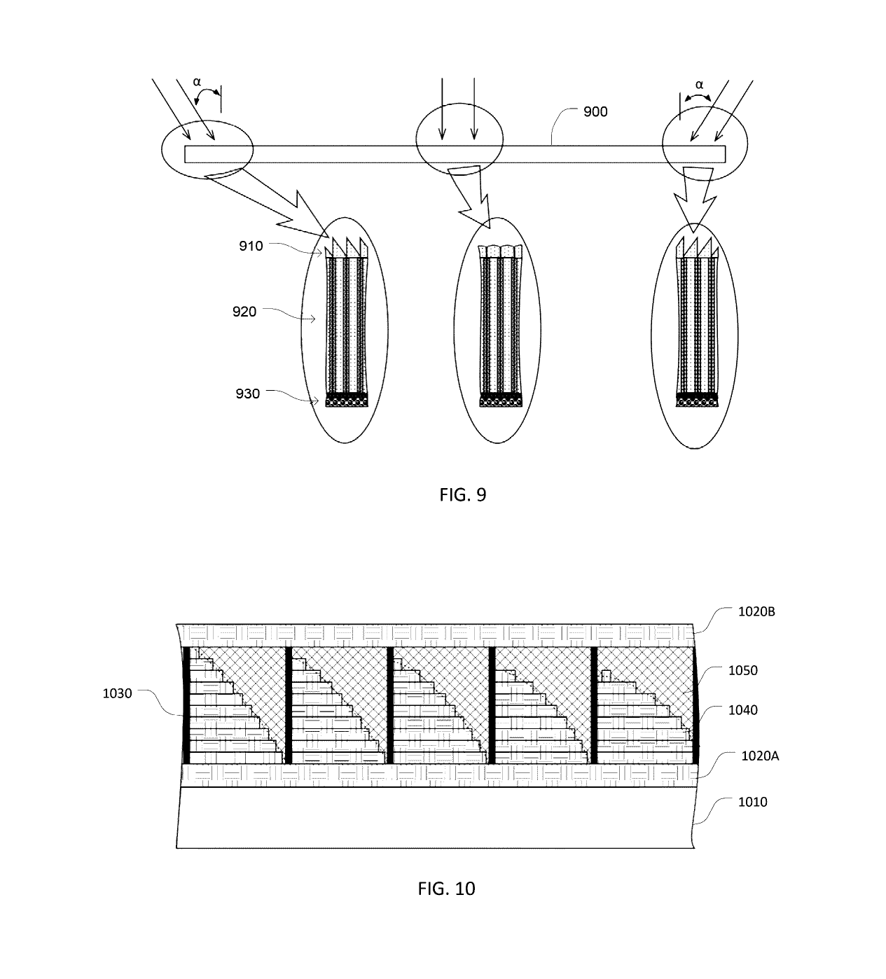

FIG. 9 illustrates a side view and partial cross sections of a bee-eye imager 900, according to an embodiment. The bee-eye imager 200 may include a lens assembly 910, a light pipe assembly 920, and an imager chip 930.

The lens assembly 910 may capture light incident on the bee-eye imager 900, and may output the captured light to the light pipe assembly 920. The lens assembly 910 may include a plurality of lenses. An edge of the lens assembly 910 may capture incident light at an angle a with respect to a normal direction of a surface of the bee-eye imager 900. In an embodiment, the angle a may be 32 degrees.

The light pipe assembly 920 may channel light from the lens assembly 910 to the CMOS imager chip 930. The light pipe assembly 920 may include a plurality of light pipes respectively coupled to the plurality of lenses in the lens assembly 910.

The imager chip 930 may convert the light from the light pipe assembly 920 into an image, such as a digital image. The imager chip 930 may include a plurality of pixels respectively corresponding to the plurality of light pipes in the light pipe assembly 930. The imager chip 930 may be a CMOS imager chip.

FIG. 10 illustrates a cross section of a bee-eye lens assembly, according to an embodiment. The bee-eye lens assembly may include a substrate 1010, a first outer layer 1020A, cell boundaries 1030, lenses 1040, filler 1050, and a second outer layer 1020B.

The substrate 1010 may be a sacrificial substrate. In an embodiment, the substrate 1010 is a planarized silicon wafer.

The first outer layer 1020A may be disposed between a plurality of cells and the substrate 1010. The first outer layer 1020A may extend across the substrate 1010, and may include a first transparent material. The first transparent material may be silicon oxide, for example. In an embodiment, the first outer layer 1020A may have a thickness of 2 micrometers.

The cell boundaries 1030 may separate the plurality of cells from one another, and may extend from the first outer layer 1020A to the second outer layer 1020B. The cell boundaries 1030 may include an optically opaque material, such as poly silicon. The optically opaque material may have a granular surface to minimize optical reflections. In an embodiment, the cell boundaries 1030 may be separated by 8 micrometers, and may each extend 10 micrometers from the first outer layer 1020A to the second outer layer 1020B.

The lenses 1040 may be respectively disposed in the plurality of cells, and may each include a plurality of layers. The lenses 1040 may each include a base layer disposed next to the first outer layer 1020A, and progressively smaller layers stacked on the base layer. In an embodiment, each lens 1040 includes 11 or fewer layers that are each 1 micrometer thick. The lenses 1040 may include a second transparent material. In an embodiment, the second transparent material is silicon dioxide.

A first side of each lens 1040 may be disposed against one of the cell boundaries 1030. Accordingly, another side of each lens 1040 may have a specific curvature defined by the progressively smaller layers.

The lenses 1040 in the lens assembly may transmit and bend light due to the curvature. Different lenses 1040 in the lens assembly may have different curvatures. For example, lenses 1040 located at an outer edge of the lens assembly may bend light at a greater angle than lenses 1040 located at a center of the lens assembly.

The fillers 1050 may fill a negative space around the lenses 1040 in each of the cells defined by the cell boundaries 1030. The fillers 1050 may include a material that has a lower index of refraction than the lenses 1040 and the first and second outer layers 1020A and 1020B. The fillers 1050 may include silicon dioxide. The fillers 1050 may be sacrificial, such that they are etched and removed during a fabrication process in order to create voids or air pockets around the lenses 1040.

The second outer layer 1020B may extend across the cells defined by the cell boundaries 1030. The second outer layer 1020B may include the same material as the first outer layer 1020A.

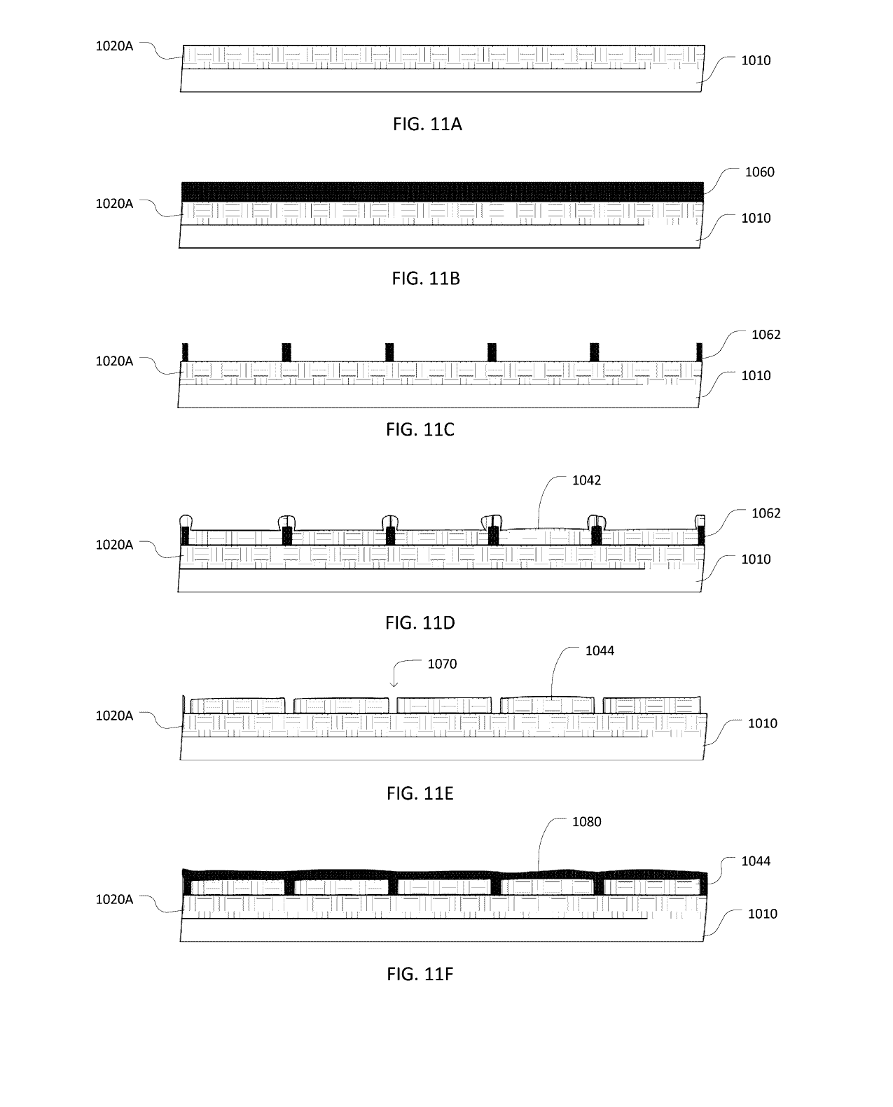

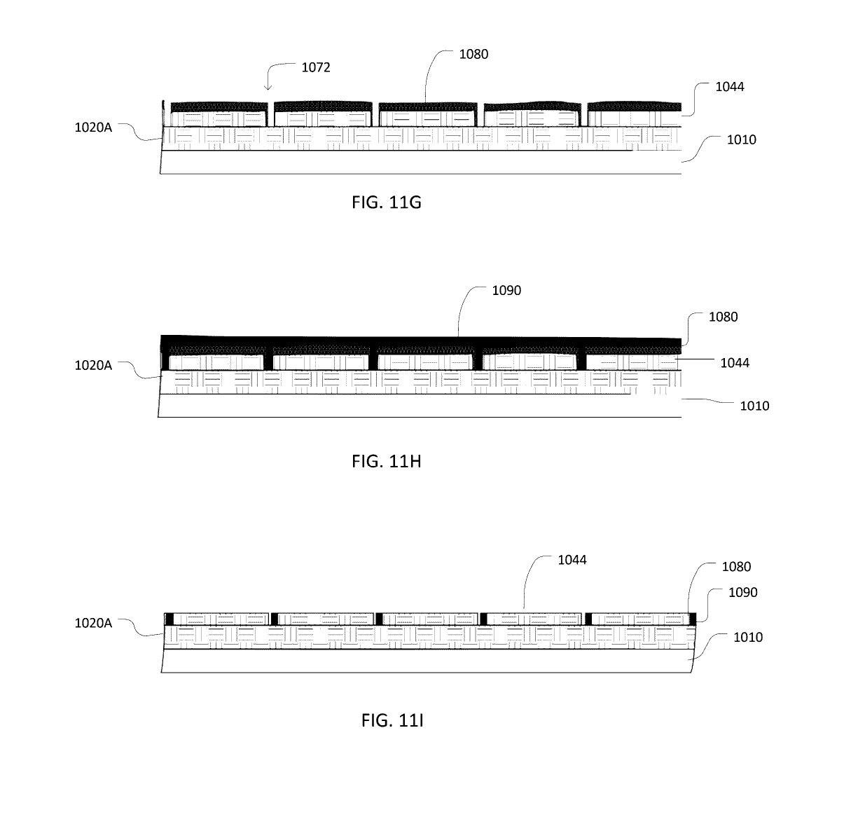

FIGS. 11A through 111 illustrate fabrication steps of a bee-eye imager, according to an embodiment.

In FIG. 11A, the substrate 1010 may be covered by the first outer layer 1020A. The substrate 1010 may be a planarized silicon wafer, and may be optically flat. The first outer layer 1020A may include a thermal oxide. In an embodiment, the first outer layer 1020A may be grown on the substrate 1010, to have a height of, for example, 2 micrometers.

The first outer layer 1020A may be subsequently polished, such that it is optically flat.

In FIG. 11B, a first photoresist layer 1060 may be deposited on the first outer layer 1020A. The first photoresist layer 1060 may be a lift-off photoresist layer.

As illustrated in FIG. 11C, first photoresist patterns 1062 may be formed by etching the first photoresist layer 1060, thereby exposing an upper surface of the first outer layer 1020A at a plurality of positions between the first photoresist patterns 1062.

In FIG. 11D, a low-temperature silicon dioxide layer 1042 may be deposited over first photoresist patterns 1062 and on the exposed surface of the first outer layer 1020A. The silicon dioxide layer 1042 may be deposited via chemical vapor deposition. The silicon dioxide layer 1042 may correspond to the material of the lenses 1040.

In FIG. 11E, the first photoresist patterns 1062 may be removed via a lift-off procedure. Excess portions of the silicon dioxide layer 1041 on top of the first photoresist patterns 1062 may also be removed, thereby forming base layers 1044. The base layers 1044 may be separated by first trenches 1070.

In FIG. 11F, a second photoresist layer 1080 may be deposited on the base layers 1044 and an upper surface of the first outer layer 1020A exposed by the first trenches 1070.

In FIG. 11G, second trenches 1072 may be etched into the second photoresist layer 1080 between the base layers 1044. The second trenches 1072 may expose the upper surface of the first outer layer 1020A.

In FIG. 11H, an optically opaque material 1090 may be deposited over the second photoresist layer 1080, thereby filling the second trenches 1070 in the second photoresist layer 1080.

Subsequently, in FIG. 11I, the optically opaque material 1090, the second photoresist layer 1080, and the base layers 1044 may be planarized. The base layers 1044 may each have a height of about 1 micrometer. Upper surfaces of the sections of the base layers 1044 and the optically opaque material 1090 may be exposed. The remaining second photoresist layer 1080 may be removed.

In an embodiment, the preceding steps of FIGS. 11A through 11I may be repeated as necessary to create multiple stacked layers in cells of a lens assembly.

FIG. 12 illustrates a detail view of a light pipe assembly, according to an embodiment. The light pipe assembly may include a plurality of light pipes that are optically isolated from one another.

Each light pipe may include a core material 1220 and a sheath material 1240. The core material 1220 may have a lower index of refraction than the sheath material 1040. In an embodiment, the sheath material 1240 may include a third material that has a greater index of refraction than the transparent materials of the first and second outer layers 1020A and 1020B, the lenses 1030, and the fillers 1050 described with respect to FIG. 10. In an embodiment, the core material 1220 includes silicon dioxide and the sheath material 1040 includes any of diamond, cubic zirconium (CZ), and zirconium oxide (ZrO₂).

An isolation material 1230 may be disposed between each light pipe, and may include an optically opaque material. The isolation material 1230 between each light pipe substantially eliminates light passing through the sheath material of one pipe from leaking or passing into the adjacent pipe.

The core material 1220 and/or the sheath material 1240 may transmit light from a lens assembly to an imager. Light that hits an interface between the core material 1220 and the sheath material 1240 may pass through the light pipe unobstructed.

In an embodiment, each light pipe includes 10 layers of the core material 1220, the sheath material 1240, and/or the isolation material 1230. The layers may include layers of the core material 1220 that are 8 micrometers wide by 10 micrometers thick, and which are sandwiched between layers of the sheath material 1240 and the isolation material 1230.

FIG. 13 illustrates a detail view of a bee-eye imager pixel area 1300, according to an embodiment.

The pixel area 1300 may receive light focused by a lens in a lens assembly and transmitted by a corresponding light pipe 1330. The pixel area 1300 may have a larger area than the diameter of the corresponding light pipe 1330. For example, the light pipe 1330 may have a diameter of approximately 7.5 micrometers, and the pixel area 1300 may have an area of 8 micrometers by 8 micrometers.

The pixel area 1300 may be divided into multiple sections, e.g., four sections. Each section may correspond to a light-sensitive photocell. Each of the sections of the pixel area 1300 may correspond to a specific color dye. For example, each section may correspond to one of a red dye, a blue dye, and a green dye. In an embodiment, the sections correspond to a Bayer filter array.

A light-sensitive photocell area 1320 may be located at a center 1310 of the pixel area 1300. The photocell area 1320 may be smaller than the pixel area 1300. For example, the pixel area 1300 may have an area of 8 micrometers by 8 micrometers, and the photocell area 1320 may be circular with a diameter between 0.5 micrometers and micrometers. The resolution of the corresponding imager may increase as the diameter of the photocell area 1320 decreases. The photocell area 1320 may overlap each of the sections of the pixel area 1300. In an embodiment, light passing through the photocell area 1320 is measured by one or more photocells corresponding to one or more photosensors, such as photoresistors, photodiodes, and/or phototransistors.

Light from the light pipe 1330 that falls outside of photocell area 1320 may be rejected by the pixel area 1300. The pixel area 1300 may register light that shines on the photocell area 1320.

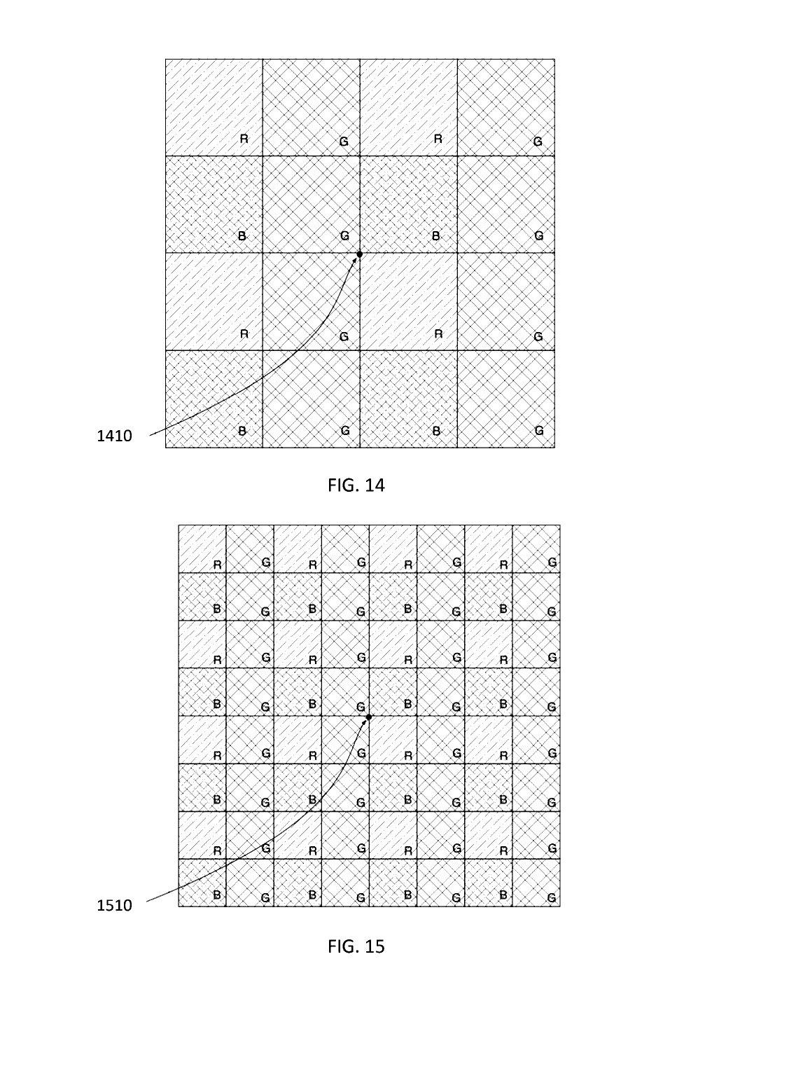

FIG. 14 illustrates a pixel area 1400 with a center 1410. The pixel area 1400 may be divided into 16 sections, each corresponding to a specific color dye. The pixel area 1400 may include four photocell areas, each corresponding to groups of four of the 16 sections. Accordingly, the pixel area 1400 may contain four pixels. In an embodiment, each section may be 2 micrometers by 2 micrometers. The pixel area 1400 may register light that falls onto any photocell area, so that there are no wasted light gathering areas. Accordingly, the pixel area 1400 may achieve maximum light sensitivity.

FIG. 15 illustrates a pixel area 1500 with a center 1510. The pixel area 1500 may be divided into 64 sections, each corresponding to a specific color dye. The pixel area 1500 may include 16 photocell areas, each corresponding to groups of four of the 64 sections, such that the pixel area 1500 may contain 16 pixels. In an embodiment, each section may be 1 micrometer by 1 micrometer.

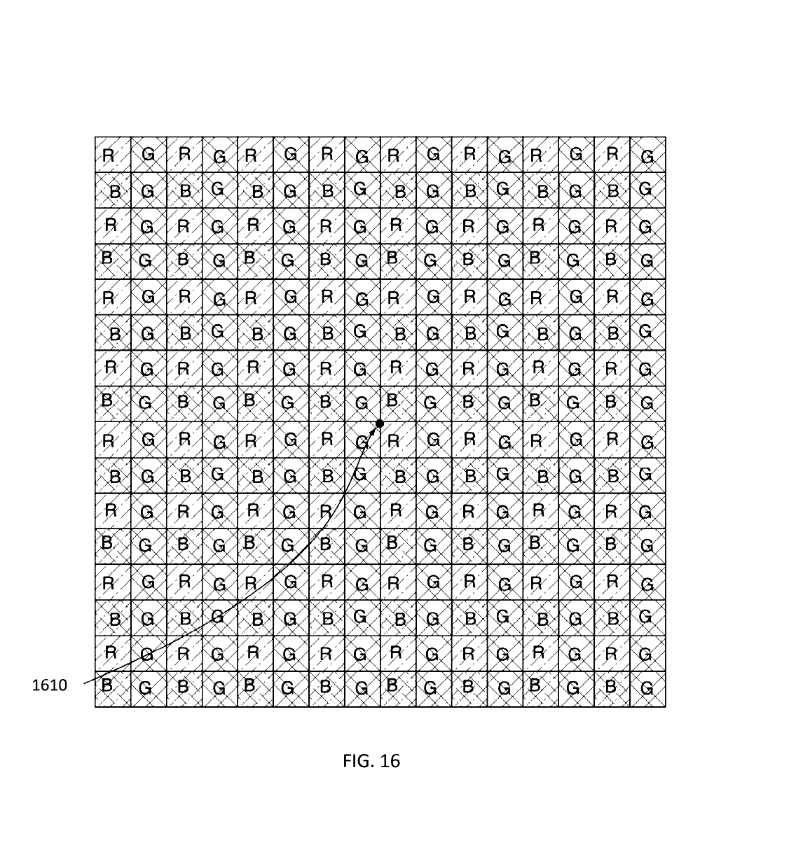

FIG. 16 illustrates a pixel area 1600 with a center 1610. The pixel area 1600 may be divided into 256 sections, each corresponding to a specific color dye. The pixel area 1500 may include 64 photocell areas, each corresponding to groups of four of the 256 sections, such that the pixel area 1600 may contain 64 pixels. In an embodiment, each section may be 0.25 micrometers by 0.25 micrometers.

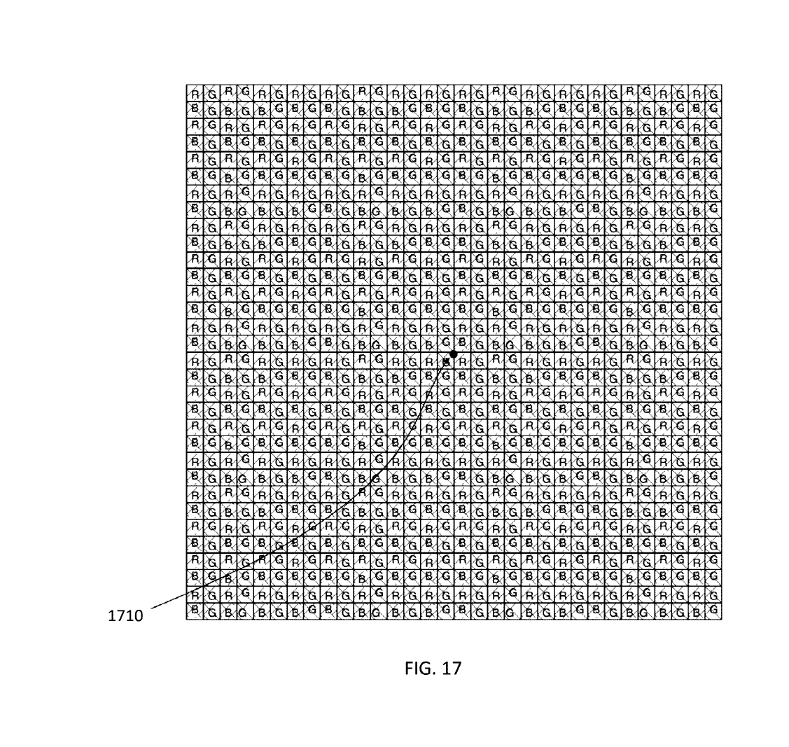

FIG. 17 illustrates a pixel area 1700 with a center 1710. The pixel area 1700 may be divided into 1024 sections, each corresponding to a specific color dye. The pixel area 1700 may include 256 photocell areas, each corresponding to groups of four of the 1024 sections, such that the pixel area 1700 may contain 256 pixels. In an embodiment, each section may be 0.0625 micrometers by 0.625 micrometers.

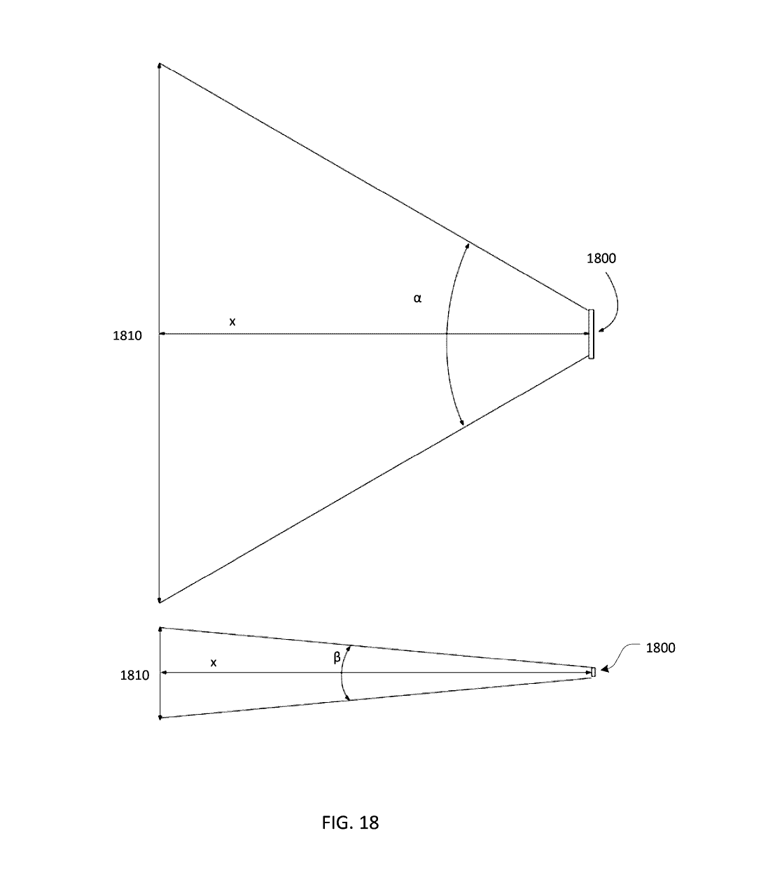

FIG. 18 illustrates horizontal and vertical fields of view of a bee-eye imager, according to an embodiment.

The bee-eye imager may have a vertical field of view corresponding to a first angle, and a horizontal field of view corresponding to a second angle.

The first and second angles may be defined with respect to a direction normal to a surface of the bee-eye imager. The bee-eye imager may have a greater vertical length than a horizontal length, and the first angle may be larger than the second angle. In an embodiment, the first angle may be 60 degrees, and the second angle may be 10.56 degrees. The bee-eye imager may have a resolution of multiple pixels per degree. For example, the resolution of the bee-eye imager may be 0.12 degrees per pixel.

FIG. 18 illustrates field of view 1810 of a bee-eye imager 1800, according to an embodiment. The bee-eye imager 1800 may sense light that enters the bee-eye imager 1800 on a rectangular surface. The field of view 1810 may also be rectangular.

The field of view 1810 may be defined by a first angle α along a first axis of the bee-eye imager 1800, and may be defined by a second angle β along a second axis of the bee-eye imager 1800. The first angle α may be greater than the second angle β, such that the field of view 1810 along the first axis is greater than the field of view 1810 along the second axis. In an embodiment, the first angle α is about 120 degrees, and the second angle β is about 21.21 degrees.

Each pixel in the bee-eye imager 1800 may have the same angular field of view along the first axis and the second axis. For example, each pixel may have a field of view of 0.12 degrees along each of the first axis and the second axis. However, the bee eye imager 1800 may include fewer pixels along the second axis than the first axis, which may cause the second angle β to be smaller than the first angle α. For example, the bee-eye imager 1800 may have 500 pixels along the first axis and 88 pixels along the second axis.

The bee eye imager 1800 may have a specific focal distance x. In an embodiment, the focal distance x may be about 35 mm, such that the bee eye imager 1800 can view 44 mm along the first axis and 7.7 mm along the second axis.

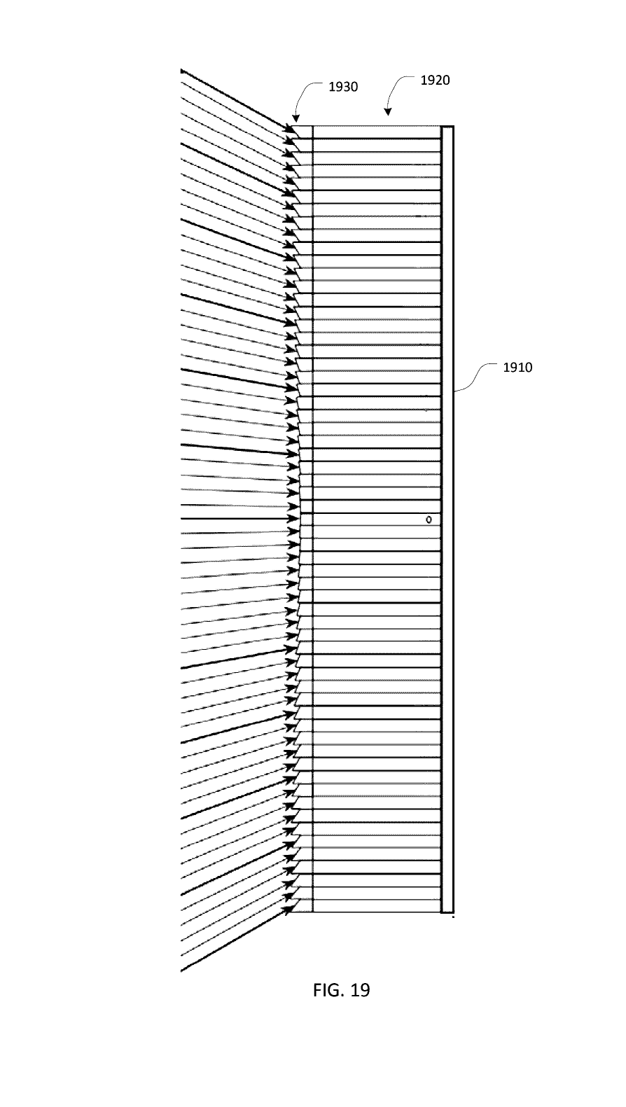

FIG. 19 illustrates a linear pixel array of a bee eye imager, according to an embodiment. In an embodiment, the linear pixel array may include a plurality of pixels arranged in a line with a specific viewing angle. For example, the linear pixel array may have 60 pixels arranged in a line with a total viewing angle of 60 degrees.

The linear pixel array may include an imager chip 1910, a light pipe assembly 1920, and a lens assembly 1930. The imager chip 1910 may include a plurality of photocells optically coupled to a plurality of light pipes in the light pipe assembly 1920. The light pipe assembly 1920 may transmit light from the lens assembly 1930 to the imager chip 1910.

The lens assembly 1930 may include a plurality of lenses that each bend light at a specific angle before transmitting the light to the light pipe assembly 1920. The individual lenses may be faceted, in a similar way to a Fresnel lens. Each lens may direct and focus a portion of the image to an individual pixel photocell in the imager chip 1910. Each faceted lens focuses its respective image through a light pipe in the light pipe assembly 1920. The lens assembly 1930 may be globally concave, but each of the lenses may be individually convex.

Lenses at the outer edge of the lens assembly 1930 may bend light at a greater angle than lenses at the center of the lens assembly 1930. In an embodiment, a lens at the center of the lens assembly 1930 transmits incident light without bending the light beam, whereas a lens at the outer edge of the lens assembly 1930 may bend incident light by 60 degrees before transmitting the light to the light pipe assembly 1920.

Each of the plurality of lenses may have a predetermined angular field of view. For example, each of the plurality of lenses may have a 1 degree field of view. FIG. 19 can be extended into and out of the page and create a 1 degree cone in the third dimension, i.e., a 1 degree steradian.

The resolution of the linear pixel array maybe proportional to a length of the light pipes in the light pipe assembly 1920. For example, the resolution is proportional to the length of the light pipe after the faceted lens and is approximately 0.5 degree for a 100 micrometer light pipe (˜focus length) with a corresponding 2 micrometer diameter photocell.

The depth of focus of the imager chip 1910 may be effectively infinite. For example, the imager chip 1910 may focus on structures that are greater than 10 focal distances from the lenses in the lens assembly 1930.

In an embodiment, imager chip 1910 may have a size of 4 mm by 0.7 mm, a depth of focus that is greater than 1 mm, and the perceived viewed image increases to a field of 10× (or 11× including itself) to a 44 mm by 7.7 mm field of view at a focal distance of 40 mm.

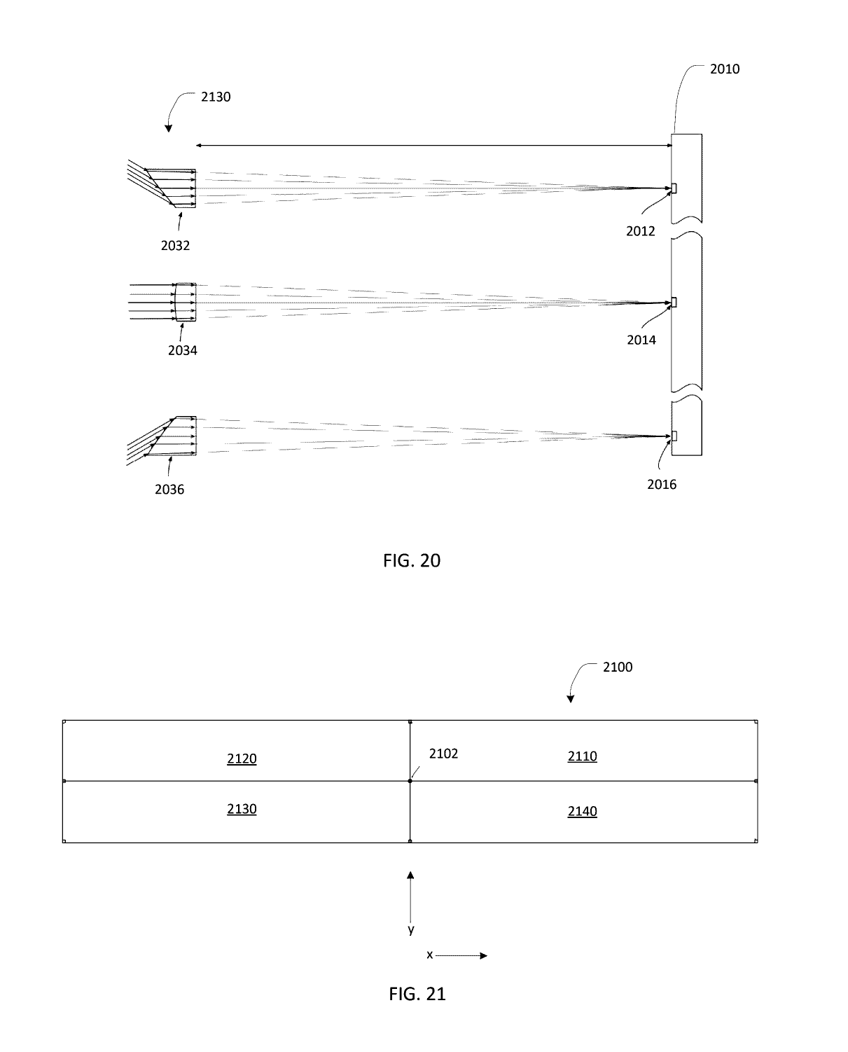

FIG. 20 illustrates a light diagram of a bee eye imager, according to an embodiment. Specifically, FIG. 20 depicts a light diagram of an imager 2010 and a lens assembly 2030.

The imager 2010 includes a plurality of photocells corresponding to respective light pipes. The plurality of photocells may include a first photocell 2012, a second photocell 2014, and a third photocell 2016. Each photocell may include a photosensor, such as any of a photoresistor, a photodiode, and a phototransistor. In an embodiment, each photocell is 0.5 to 2 micrometers by 0.5 to 2 micrometers, and/or has a diameter of 0.5 to 2 micrometers.

The lens assembly 2030 may include a plurality of lenses, such as a first lens 2032, a second lens 2034, and a third lens 2036. Each of the lenses 2032, 2034, and 2036 may focus light on a respective photocell 2012, 2014, and 2016 through a respective light pipe (not shown). The depth of field may be greater than 10 focal lengths of each of the lenses 2032, 2034, and 2036. Accordingly, the bee eye imager can be used to image objects that are over 10 focal lengths away from the lens assembly 2030.

The lenses 2032, 2034, and 2036 may receive incident light at an angle. Lenses located toward an outer edge of the lens assembly 2030 may receive incident light at a greater angle than lenses located toward a center axis of the lens assembly 2030. For example, the first lens 2032 may accept light at a 330 degree angle, the second lens 2034 may accept light at a 0 degree angle, and the third lens 2036 may accept light at a 30 degree angle.

The lenses 2032, 2034, and 2036 in the lens assembly 2030 may have the same focal length. For example, the focal length may be 100 micrometers.

The position of respective lenses and photocells can be described on an x-axis. In an embodiment, the first lens 2032 has a position of x=250, the second lens 2034 has a position of x=0, and the third lens 2036 has a position of x=−250. Similarly, the first photocell 2012 may have a position of x=250, the second lens 2014 may have a position of x=0, and the third photocell may have a position of x=−250. The angle at which a lens accepts light may be proportional to the x position of the lens.

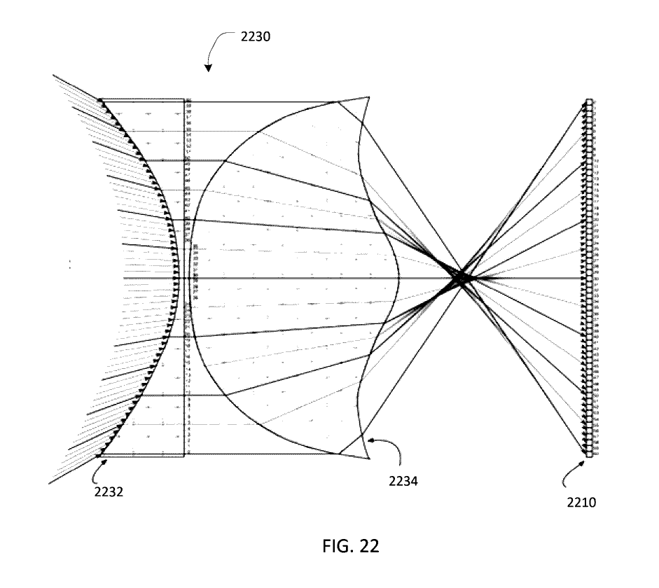

FIG. 21 illustrates pixel positioning nomenclature of a bee eye imager 2100, according to an embodiment.

The bee eye imager 2100 may include a plurality of pixels arranged in a rectangular grid along an x direction and a y direction. Each pixel may have a respective position in the rectangular grid defined by x and y. For a pixel at the center of the bee eye imager, x may be 0 and y may be 0. For a pixel in a first quadrant 2110 of the bee eye imager, x and y may be positive integers. For a pixel in a second quadrant 2120, x may be a negative integer and y may be a positive integer. For a pixel in a third quadrant 2130, x and y may be negative integers. For a pixel in a fourth quadrant 2140, x may be a positive integer and y may be a negative integer. In an embodiment, x may range from [−250, 250], and y may range from [−44, 44], such that the bee eye imager 2100 may have a length of 500 pixels and a height of 88 pixels.

FIG. 22 illustrates an imager 2210 and a lens assembly 2230, according to an embodiment.

The lens assembly 2230 may include first and second continuous lenses 2232 and 2234. The first continuous lens 2232 may be a concave lens, which may convert light from a 60 degree field of view to parallel light rays. The second lens 2234 may be a concave lens that may focus the parallel light rays to pixels on the imager.

The lens assembly 2230 of FIG. 22 may have a greater thickness than the lens assembly 1930 and light pipe assembly 1920 of FIG. 19, for example, but may utilize the same design rules. For example, the lens assembly 2230 may have a thickness of 830 micrometers, and the lens assembly 1930 and light pipe assembly 1920 of FIG. 19 may have a collective thickness of 120 micrometers using similar design rules. In some applications, the thinner lens assembly 1930 and 1920 may be preferred over the lens assembly 2230.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

What is claimed is:

1. An imager, comprising:
a lens assembly including a plurality of lenses;
an imager chip including a plurality of photocells; and
a light pipe assembly including a plurality of light pipes optically coupled between the plurality of lenses and the plurality of image sensors.

2. The imager of claim 1, wherein the lens assembly further includes:
a plurality of dividers between the plurality of lenses, the plurality of dividers including an optically opaque material.

3. The imager of claim 2, wherein each of the plurality of lenses includes a stack of transparent layers, the transparent layers being stacked in an order of decreasing size.

4. The imager of claim 1, wherein each of the plurality of lenses bends and focuses light at one of the plurality of photocells, and
wherein a first lens at an edge of the light pipe assembly bends light at a greater angle than a second lens at a center of the light pipe assembly.

5. The imager of claim 1, wherein the plurality of photocells are spaced apart from the plurality of lenses by a focal distance of the plurality of lenses.

6. The imager of claim 1, wherein the plurality of photocells include complementary metal oxide semiconductor (CMOS) image sensors.

7. The imager of claim 1, wherein the light pipe assembly further includes:
an isolation material disposed between the light pipes.

8. The imager of claim 7, wherein each of the plurality of light pipes includes a core material surrounded by a sheath material, the core material having a lower index of refraction than the sheath material.

9. The imager of claim 1, wherein the imager chip further includes a plurality of pixels, each of the plurality of pixels being divided into a plurality of color areas, and
wherein each of the photocells corresponds to at least one of the color areas.

10. The imager of claim 9, wherein each of the plurality of light pipes is coupled to at least one of the photocells.

11. An imaging device, comprising:
a needle including a tip;
an imager disposed on the needle, the imager including a lens assembly, an imager chip, and a light pipe assembly, the lens assembly including a plurality of lenses, the imager chip including a plurality of image sensors, and the light pipe assembly including a plurality of light pipes optically coupled between the plurality of lenses and the plurality of image sensors; and
a syringe coupled to the needle.

12. The imaging device of claim 11, further comprising:
a light-emitting diode (LED) provided proximate to the imager; and
a bypass capacitor disposed inside of the needle and proximate to the imager.

13. The imaging device of claim 11, wherein the lens assembly further includes:
a plurality of dividers between the plurality of lenses, the plurality of dividers including an optically opaque material,
wherein each of the plurality of lenses includes a stack of transparent layers, the transparent layers being stacked in an order of decreasing size.

14. The imaging device of claim 11, wherein each of the plurality of lenses bends and focuses light toward one of the plurality of photocells, and
wherein a first lens at an edge of the light pipe assembly bends light at a greater angle than a second lens at a center of the light pipe assembly.

15. The imaging device of claim 11, wherein the plurality of image sensors are spaced apart from the plurality of lenses by a focal distance of the plurality of lenses.

16. The imaging device of claim 11, wherein the light pipe assembly further includes:
an isolation material disposed between the light pipes,
wherein each of the plurality of light pipes includes a core material surrounded by a sheath material, the sheath material having a larger index of refraction than the core material.

17. The imaging device of claim 11, wherein the imager chip further includes a plurality of pixels, each of the plurality of pixels being divided into a plurality of color areas,
wherein each of the photocells corresponds to at least one of the color areas, and
wherein each of the plurality of light pipes is coupled to at least one of the photocells.
